(19) United States
(12) Patent Application Publication
(10) Pub. No.: US 2016/0139252 A1
(43) Pub. Date: May 19, 2016

(54) ULTRASOUND DIAGNOSTIC DEVICE, METHOD FOR GENERATING ACOUSTIC RAY SIGNAL OF ULTRASOUND DIAGNOSTIC DEVICE, AND PROGRAM FOR GENERATING ACOUSTIC RAY SIGNAL OF ULTRASOUND DIAGNOSTIC DEVICE

(71) Applicant: FUJIFILM CORPORATION, Tokyo (JP)

(72) Inventor: Kimito KATSUYAMA, Kanagawa (JP)

(21) Appl. No.: 14/952,926

(22) Filed: Nov. 26, 2015

Related U.S. Application Data
(63) Continuation of application No. PCT/JP2014/061319, filed on Apr. 22, 2014.

(30) Foreign Application Priority Data
May 31, 2013 (JP) 2013-116021

(51) Int. Cl. G01S 7/52; A61B 8/14; A61B 8/00; G10K 11/34

(52) U.S. Cl. CPC G01S 7/52047; G10K 11/346; A61B 8/14; A61B 8/4488

(57) ABSTRACT

An ultrasound beam is transmitted to an inspection target by determining an opening of a positive phase and an opening of a negative phase corresponding to the opening of the positive phase in advance and inverting the phases with a group of a predetermined number of elements of a probe as each opening, and an ultrasound echo signal generated by interaction between the ultrasound beam and the inspection target is received through the plural elements of the probe. Then, two or more pieces of element data including receiving time information in each element, which are generated by receiving ultrasound echo signals generated for at least two or more overlapping target regions, are stored, and an acoustic ray signal in one scanning line is generated by overlapping the stored element data based on the receiving time information in each element.

### CROSS-REFERENCE TO RELATED APPLICATIONS

This application is a continuation application of International Application No. PCT/JP2014/061319, filed on Apr. 22, 2014, which is incorporated herein by reference in its entirety. Further, this application claims priority from Japanese Patent Application No. 2013-116021, filed on May 31, 2013, the disclosure of which is incorporated by reference herein in its entirety.

### BACKGROUND

1. Technical Field

The present disclosure relates to an ultrasound diagnostic device, an acoustic ray signal generation method of an ultrasound diagnostic device, and a storage medium for storing an acoustic ray signal generation program of an ultrasound diagnostic device.

2. Description of the Related Art

As a technique for reducing side lobes in ultrasound diagnosis or for improving the azimuth resolution, a technique disclosed in “KIURA Nobuyuki, “Harmonic Imaging”, Japanese Society of Radiological Technology Magazine, P 350-356” or JP1983-44372A (JP-558-44372A) has been proposed.

“KIURA Nobuyuki, “Harmonic Imaging”, Japanese Society of Radiological Technology Magazine, P 350-356” proposes a method for solving the disadvantages of a filtering method of separating fundamental wave components and secondary harmonic components from each other using a secondary harmonic detection filter and extracting only the secondary harmonic components to perform imaging. Specifically, two ultrasound transmissions are performed consecutively in the same direction with the phases of the first and second transmission waves being inverted. Fundamental wave components of the reception waves of two times by the transmission waves of two times have inverted phases, but the secondary harmonic components have the same phase. Therefore, doubled secondary harmonic components are obtained by removing the fundamental wave components by adding up the fundamental wave components and the secondary harmonic components of the reception waves of two times. Therefore, it is possible to solve a reduction in distance resolution that is a disadvantage of the filter method.

On the other hand, JP1983-44372A (JP-S58-44372A) proposes a technique for improving the azimuth resolution and the contrast resolution by sequentially adding up reception signals based on the transmission and reception of plural different openings.

In the technique disclosed in “KIURA Nobuyuki, “Harmonic Imaging”, Japanese Society of Radiological Technology Magazine, P 350-356”, however, the number of frames is halved since imaging based on two ultrasound transmissions is performed. Accordingly, the time resolution is reduced.

On the other hand, in the technique disclosed in JP1983-44372A (JP-558-44372A), it is assumed that spherical waves converge and diverge by regarding a focus formed by transmission focusing as a single sound source. However, since secondary side lobes are formed by the transmission focusing, the side lobes are reflected on the reception signals. This degrades the image quality.

### SUMMARY

The present disclosure has been made in view of the above situation, and provides an ultrasound diagnostic device capable of obtaining a high-quality ultrasound image by reducing side lobes, an acoustic ray signal generation method of an ultrasound diagnostic device, and a non-transitory storage medium for storing an acoustic ray signal generation program of an ultrasound diagnostic device.

A first aspect of the present disclosure is an ultrasound diagnostic device including: a probe including plural elements for generating ultrasound waves and receiving ultrasound waves reflected from an inspection target; a transmission unit that transmits ultrasound waves from the plural elements such that an ultrasound beam is transmitted to the inspection target by determining a group of a predetermined number of elements of the probe as openings, determining in advance an opening of a positive phase and an opening of a negative phase corresponding to the opening of the positive phase and inverting phases thereof; a receiving unit that receives an ultrasound echo signal generated by interaction between the ultrasound beam and the inspection target, through the plural elements of the probe; an element data storage unit that stores two or more sets of element data including receiving time information in each element, the two or more sets of element data being generated by transmitting the ultrasound beam from openings having different phases and having different elements at centers, in each of at least two or more overlapping target regions in the inspection target using the transmission unit, and receiving the ultrasound echo signal generated for each of the at least two or more overlapping target regions in the inspection target by the ultrasound beam, using the receiving unit; and a generation unit that generates an acoustic ray signal in one scanning line by overlapping the element data stored in the element data storage unit.

According to the ultrasound diagnostic device of the first aspect, the probe includes plural elements for generating ultrasound waves and receiving ultrasound waves reflected from the inspection target.

The transmission unit transmits ultrasound waves from the plural elements such that the ultrasound beam is transmitted to the inspection target by determining the opening of the positive phase and the opening of the negative phase in advance and inverting the phases with a group of a predetermined number of elements of the probe as each opening. That is, ultrasound waves having inverted phases are transmitted from the opening of the positive phase and the opening of the negative phase.

The receiving unit receives an ultrasound echo signal, which is generated by the interaction between the ultrasound beam and the inspection target, through the plural elements of the probe.

Two or more sets of element data including the receiving time information in each element that are generated by transmitting the ultrasound beam from openings, which have different phases and have different elements at centers, in each of at least two or more overlapping target regions in the inspection target using the transmission unit and receiving the ultrasound echo signal, which is generated for each of at least two or more overlapping target regions in the inspection target by the ultrasound beam, using the receiving unit are stored in the element data storage unit.

The generation unit generates an acoustic ray signal in one scanning line by overlapping the element data stored in the element data storage unit. That is, since only the secondary harmonic components are extracted by performing overlapping by adding up the element data in the opening of the positive phase and the element data in the opening of the negative phase, it is possible to generate an acoustic ray signal in which the influence of side lobes is suppressed. Therefore, it is possible to obtain a high-quality ultrasound image by reducing side lobes.

The transmission unit may transmit ultrasound waves from the plural elements by determining the number of the openings as an even number, determining in advance openings that are located symmetrically with respect to the scanning line as the opening of the positive phase and the opening of the negative phase, inverting the phases thereof and transmitting the ultrasound beam from each of the openings.

Alternatively, the transmission unit may transmit ultrasound waves from the plural elements by determining the number of the openings as an odd number, inverting the phases thereof and transmitting the ultrasound beam from each of the opening of the positive phase and the opening of the negative phase, and the generation unit may generate the acoustic ray signal by overlapping the element data stored in the element data storage unit based on the receiving time information and a coefficient that is set in advance for each opening such that sound pressure from a noted point is the same.

The generation unit may include: a delay time calculation section that calculates a delay time of the two or more sets of the element data; and an overlapping processing section that overlaps two or more sets of non-processed element data so that times thereof match on a receiving time and absolute positions of the elements of the probe that have received the ultrasound echo signal match, based on the delay time calculated by the delay time calculation section, the receiving time information, and positions of the elements of the probe that has received the ultrasound echo signal.

The transmission unit may transmit ultrasound waves from the plural elements such that the opening of the positive phase and the opening of the negative phase adjacent to each other alternately transmit ultrasound beams.

On the other hand, a second aspect of the present disclosure is an acoustic ray signal generation method of an ultrasound diagnostic device including a transmission unit that transmits ultrasound waves from plural elements of a probe for generating ultrasound waves and receiving ultrasound waves reflected from the inspection target, such that the ultrasound beam is transmitted to an inspection target by determining a group of a predetermined number of the elements of the probe as openings, determining in advance an opening of a positive phase and an opening of a negative phase corresponding to the opening of the positive phase and inverting phases thereof, a receiving unit that receives an ultrasound echo signal generated by interaction between the ultrasound beam and the inspection target, through the plural elements of the probe, and in an element data storage unit, the method including: transmitting, by the transmission unit, an ultrasound beam from openings having different phases and having different elements at centers, in each of at least two or more overlapping target regions in the inspection target; receiving, by the receiving unit, an ultrasound echo signal generated for each of the at least two or more overlapping target regions in the inspection target; storing two or more sets of element data generated by receiving the ultrasound echo signal in the element data storage unit, the two or more set of element data including receiving time information in each element; and generating an acoustic ray signal in one scanning line by overlapping the element data stored in the element data storage unit.

According to the acoustic ray signal generation method of an ultrasound diagnostic device of the second aspect, the ultrasound beam is transmitted from the openings, which have different phases and have different elements at centers, in each of at least two or more overlapping target regions in the inspection target using the transmission unit that transmits ultrasound waves from the plural elements such that the ultrasound beam is transmitted to the inspection target by determining the opening of the positive phase and the opening of the negative phase in advance and inverting the phases with a group of a predetermined number of elements of the probe as each opening, the probe including a plural elements for generating ultrasound waves and receiving ultrasound waves reflected from the inspection target. That is, ultrasound waves having inverted phases are transmitted from the opening of the positive phase and the opening of the negative phase.

Then, the ultrasound echo signal generated for each of at least two or more overlapping target regions in the inspection target is received using the receiving unit that receives an ultrasound echo signal, which is generated by the interaction between the ultrasound beam and the inspection target, through the plural elements of the probe.

Then, two or more sets of element data including the receiving time information in each element, which are generated by receiving the ultrasound echo signal, are stored in the element data storage unit.

Then, based on the receiving time information in each element, an acoustic ray signal in one scanning line is generated by overlapping the element data stored in the element data storage unit. That is, since only the secondary harmonic components are extracted by performing overlapping by adding up the element data in the opening of the positive phase and the element data in the negative phase, it is possible to generate an acoustic ray signal in which the influence of side lobes is suppressed. Therefore, it is possible to obtain a high-quality ultrasound image by reducing side lobes.

The ultrasound waves may be transmitted from the plural elements by determining the number of the openings as an even number, determining in advance openings that are located symmetrically with respect to the scanning line as the opening of the positive phase and the opening of the negative phase, inverting the phases thereof and transmitting the ultrasound beam from each of the openings.

Alternatively, the ultrasound waves may be transmitted from the plural elements by inverting phases of the openings of the positive phase and the opening of the negative phase in the transmission unit having an odd number of the openings, and the acoustic ray signal may be generated by overlapping the element data stored in the element data storage unit based on the receiving time information and a coefficient that is set in advance for each opening such that sound pressure from a noted point is the same.

The generation of the acoustic ray signal may further include: calculating a delay time of the two or more sets of element data; and overlapping two or more sets of non-processed element data so that times thereof match on a receiving time and absolute positions of the elements of the probe that have received the ultrasound echo signal match, based on the calculated delay time, the receiving time information, and positions of the elements of the probe that have received the ultrasound echo signal.

Ultrasound waves may be transmitted from the plural elements such that the opening of the positive phase and the opening of the negative phase adjacent to each other alternately transmit ultrasound beams.

In addition, a third aspect of the present disclosure is a non-transitory storage medium that stores a program causing a computer to execute an acoustic ray signal generation processing of an ultrasound diagnostic device including a transmission unit that transmits ultrasound waves from plural elements of a probe for generating ultrasound waves and receiving ultrasound waves reflected from the inspection target, such that the ultrasound beam is transmitted to an inspection target by determining a group of a predetermined number of the elements of the probe as openings, determining in advance an opening of a positive phase and an opening of a negative phase corresponding to the opening of the positive phase and inverting phases thereof, a receiving unit that receives an ultrasound echo signal generated by interaction between the ultrasound beam and the inspection target, through the plural elements of the probe, and in an element data storage unit, the processing including: transmitting, by the transmission unit, an ultrasound beam from openings having different phases and having different elements at centers, in each of at least two or more overlapping target regions in the inspection target; receiving, by the receiving unit, an ultrasound echo signal is generated for each of the at least two or more overlapping target regions in the inspection target; storing two or more sets of element data generated by receiving the ultrasound echo signal in the element data storage unit, the two or more sets of element data including receiving time information in each element; and generating an acoustic ray signal in one scanning line by overlapping the element data stored in the element data storage unit.

According to the third aspect of the present disclosure, the ultrasound beam is transmitted from the openings, which have different phases and have different elements at centers, in each of at least two or more overlapping target regions in the inspection target using the transmission unit that transmits ultrasound waves from the plural elements such that the ultrasound beam is transmitted to the inspection target by determining the opening of the positive phase and the opening of the negative phase in advance and inverting the phases with a group of a predetermined number of elements of the probe as each opening, the probe including a plural elements for generating ultrasound waves and receiving ultrasound waves reflected from the inspection target. That is, ultrasound waves having inverted phases are transmitted from the opening of the positive phase and the opening of the negative phase.

Then, the ultrasound echo signal generated for each of at least two or more overlapping target regions in the inspection target is received using the receiving unit that receives an ultrasound echo signal, which is generated by the interaction between the ultrasound beam and the inspection target, through the plural elements of the probe.

Then, two or more sets of element data including the receiving time information in each element, which are generated by receiving the ultrasound echo signal, are stored in the element data storage unit.

Then, based on the receiving time information in each element, an acoustic ray signal in one scanning line is generated by overlapping the element data stored in the element data storage unit. That is, since only the secondary harmonic components are extracted by performing overlapping by adding up the element data in the opening of the positive phase and the element data in the negative phase, it is possible to generate an acoustic ray signal in which the influence of side lobes is suppressed. Therefore, it is possible to obtain a high-quality ultrasound image by reducing side lobes.

The ultrasound waves may be transmitted from the plural elements by determining the number of the openings as an even number, determining in advance openings that are located symmetrically with respect to the scanning line as the opening of the positive phase and the opening of the negative phase, inverting phases thereof and transmitting the ultrasound beam from each of the openings.

Alternatively, ultrasound waves may be transmitted from the plural elements by inverting phases of the opening of the positive phase and the opening of the negative phase of the transmission unit having an odd number of the openings, and the acoustic ray signal may be generated by overlapping the element data stored in the element data storage unit based on the receiving time information and a coefficient that is set in advance for each opening such that sound pressure from a noted point is the same.

The generation of the acoustic ray signal may further include: calculating a delay time of the two or more sets of element data; and overlapping two or more sets of non-processed element data so that times thereof match on a receiving time and absolute positions of the elements of the probe that have received the ultrasound echo signal match, based on the calculated delay time, the receiving time information, and positions of the elements of the probe that have received the ultrasound echo signal.

Ultrasound waves may be transmitted from the plural elements such that the opening of the positive phase and the opening of the negative phase adjacent to each other alternately transmit ultrasound beams.

As described above, in the present disclosure, it is possible to obtain a high-quality ultrasound image by reducing side lobes.

### BRIEF DESCRIPTION OF THE DRAWINGS

FIG. 1 is a block diagram showing the schematic configuration of an ultrasound diagnostic device according to the present embodiment.

FIG. 2 is a block diagram showing the configuration of an element data processing unit.

FIG. 3 shows cases of transmitting an ideal ultrasound beam from an element, in which (1) shows a case of transmitting an ideal ultrasound beam from an element located immediately above the reflection point of the subject, (3) shows a case of transmitting an ideal ultrasound beam from an element that is not located immediately above the reflection point of the subject, and (2) and (4) show the obtained element data, respectively.

FIG. 4 shows cases of transmitting an actual ultrasound beam from an element, in which (1) shows a case of transmitting an actual ultrasound beam from an element located immediately above the reflection point of the subject, (3) shows a case of transmitting an actual ultrasound beam from an element that is not located immediately above the reflection point of the subject, and (2) and (4) show the obtained element data, respectively.

FIG. 5A shows the distance of the transmission path of the ultrasound beam and the distance of the receiving path, in which (1) shows a case of a true reflected ultrasound echo, and (2) shows a case of a ghost reflection signal.

FIG. 5B shows: (1) element data obtained in a plural elements, and (2) their delay time.

FIG. 6 shows an exemplary flow of overlapping processing performed by a overlapping processing section, in which (1), (2) and (3) show element data obtained in a plural elements, their delay time, and the overlapping state of element data in a case of a true signal, (4), (5) and (6) show element data obtained in a plural elements, their delay time, and the overlapping state of element data in a case of a ghost, and (7) and (8) show the overlapping state of element data corresponding to a plural elements and the result, respectively.

FIG. 7A is a schematic diagram for explaining the transmission of a transmission beam that is performed while shifting the transmission opening by one element at a time and shifting the transmission focusing line by one line at a time.

FIG. 7B is a diagram showing an example of a pair of positive and negative transmission openings having inverted phases.

FIG. 8A is a diagram for explaining the addition of a pair of sets of positive and negative element data having inverted phases and the overlapping of the element data.

FIG. 8B is a diagram for explaining the addition of a pair of sets of positive and negative element data having inverted phases and the overlapping of the element data.

FIG. 9 is a schematic diagram showing the addition of a pair of sets of positive and negative transmission openings having inverted phases and the overlapping of the element data.

FIG. 10 is a block diagram showing a main part of the ultrasound diagnostic device according to the embodiment of the present disclosure along the process flow.

FIG. 11 is a schematic diagram showing the overlapping of positive and negative element data when the number of transmission openings is odd.

### DETAILED DESCRIPTION

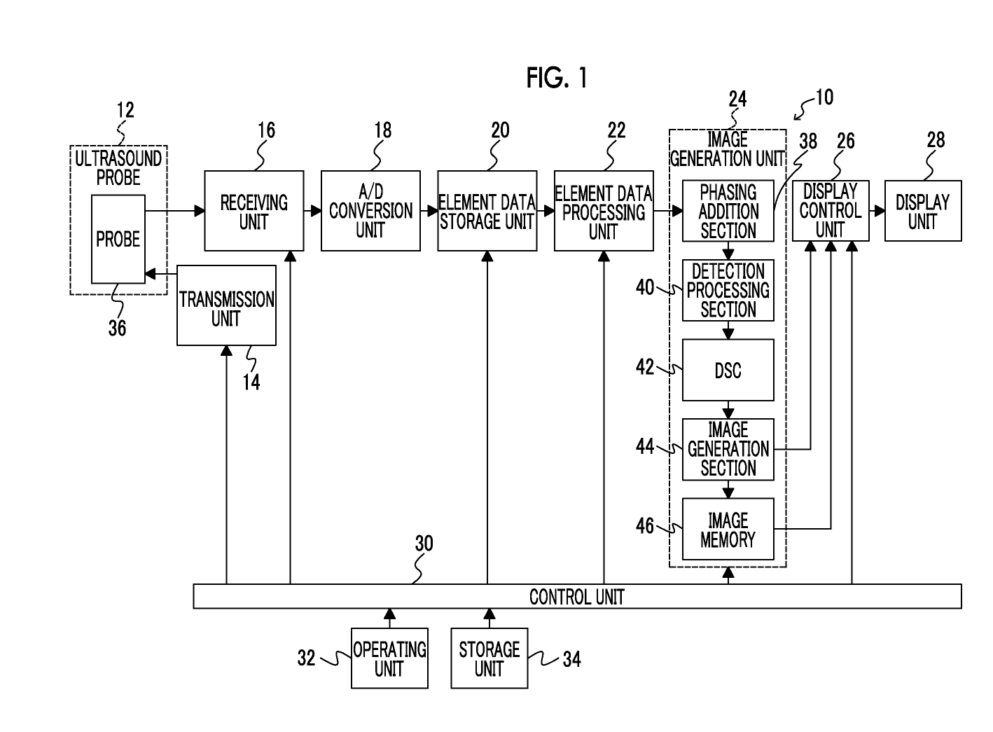

Hereinafter, an example of the present embodiment will be described with reference to each diagram. FIG. 1 is a block diagram showing the schematic configuration of an ultrasound diagnostic device according to the present embodiment.

As shown in FIG. 1, an ultrasound diagnostic device 10 includes an ultrasound probe 12, a transmission unit 14 and a receiving unit 16 that are connected to the ultrasound probe 12, an A/D conversion unit 18, an element data storage unit 20, an element data processing unit 22, an image generation unit 24, a display control unit 26, a display unit 28, a control unit 30, an operating unit 32, and a storage unit 34.

The ultrasound probe 12 has a probe 36 that is used in a normal ultrasound diagnostic device. The probe 36 has plural elements, that is, ultrasound transducers arranged in a one-dimensional or two-dimensional array. When capturing an ultrasound image of a subject, each of the ultrasound transducers transmits an ultrasound beam to the subject according to a driving signal supplied from the transmission unit 14, and receives an ultrasound echo from the subject and outputs a reception signal. In the present embodiment, each of a predetermined number of ultrasound transducers that form a set of the plural ultrasound transducers of the probe 36 generates each component of one ultrasound beam, and a set of a predetermined number of ultrasound transducers generate one ultrasound beam to be transmitted to the subject.

For example, each ultrasound transducer is formed by an element (transducer) in which electrodes are formed at both ends of the piezoelectric body formed of piezoelectric ceramic represented by lead zirconate titanate (PZT), a polymer piezoelectric element represented by polyvinylidene fluoride (PVDF), piezoelectric single crystal represented by lead magnesium niobate-lead titanate (PMN-PT), or the like. That is, the probe 36 is a transducer array in which plural transducers are arranged in a one-dimensional or two-dimensional array as plural ultrasound elements.

When a pulsed or continuous-wave voltage is applied to the electrodes of such a transducer, the piezoelectric body expands and contracts to generate pulsed or continuous-wave ultrasound waves from each transducer. By combination of these ultrasound waves, an ultrasound beam is formed. In addition, the respective transducers expand and contract by receiving the propagating ultrasound waves, thereby generating electrical signals. These electrical signals are output as reception signals of the ultrasound waves.

The transmission unit 14 includes, for example, plural pulsers. Based on a transmission delay pattern selected according to the control signal from the control unit 30, the transmission unit 14 adjusts the amount of delay of the driving signal of each ultrasound element so that the ultrasound waves transmitted from a set of a predetermined number of ultrasound transducers (hereinafter, referred to as ultrasound elements) in the probe 36 form one ultrasound beam, and supplies the adjusted driving signals to the plural ultrasound elements that form a set.

According to the control signal from the control unit 30, the receiving unit 16 receives an ultrasound echo, which is generated by the interaction between the ultrasound beam and the subject, from the subject using each ultrasound element of the probe 36, amplifies and outputs the reception signal, that is, an analog element signal for each ultrasound element, and supplies the amplified analog element signal to the A/D conversion unit 18.

The A/D conversion unit 18 is connected to the receiving unit 16, and converts the analog element signal supplied from the receiving unit 16 into digital element data. The A/D conversion unit 18 supplies the A/D-converted digital element data to the element data storage unit 20.

The element data storage unit 20 stores the digital element data output from the A/D conversion unit 18 in a sequentially manner. In addition, the element data storage unit 20 stores information regarding the frame rate input from the control unit 30 (for example, parameters indicating the depth of the reflection position of an ultrasound wave, the density of scanning lines, and a field-of-view width) so as to be associated with the above digital element data (hereinafter, simply referred to as element data).

When inspecting at least two or more target regions overlapping each other in target regions on the position coordinates of two or more dimensions according to the control signal from the control unit 30, the element data storage unit 20 stores two or more sets of element data, which have been generated for each of the two or more target regions from the ultrasound echo received by the receiving unit 16, for each of the two or more target regions. The element data stored in the element data storage unit 20 is two or more sets of element data including the receiving time information in each element.

The element data processing unit 22 generates element data after overlapping processing (hereinafter, referred to as processed element data) by overlapping two or more sets of element data generated for each of two or more target regions stored in the element data storage unit 20 (hereinafter, referred to as non-processed element data) so as to match the time on the receiving time under the control of the control unit 30. The details of the element data processing unit 22 will be described later.

Under the control of the control unit 30, the image generation unit 24 generates an acoustic ray signal (reception data) from the processed element data supplied from the element data processing unit 22, and generates an ultrasound image from the acoustic ray signal. Specifically, the image generation unit 24 includes a phasing addition section 38, a detection processing section 40, a DSC 42, an image generation section 44, and an image memory 46.

The phasing addition section 38 performs reception focusing processing by selecting one reception delay pattern from plural reception delay patterns stored in advance according to the receiving direction set by the control unit 30, applying each delay to the element data based on the selected reception delay pattern, and adding up the results. Through this reception focusing processing, reception data (acoustic ray signal) with narrowed focus of the ultrasound echo is generated. The phasing addition section 38 supplies the generated reception data to the detection processing section 40.

The detection processing section 40 generates B-mode image data, which is tomographic image information regarding tissue within the subject, by correcting the attenuation due to the distance according to the depth of the reflection position of the ultrasound wave and then performing envelope detection processing for the reception data generated by the phasing addition section 38.

The digital scan converter (DSC) 42 converts the B-mode image data generated by the detection processing section 40 into image data according to the normal television signal scanning method (raster conversion).

The image generation section 44 generates B-mode image data to be supplied for inspection or display by performing various kinds of required image processing, such as gradation processing, on the B-mode image data input from the DSC 42, and outputs the generated B-mode image data for inspection or display to the display control unit 26 in order to display it or stores the generated B-mode image data for inspection or display in the image memory 46.

The image memory 46 temporarily stores the B-mode image data for inspection generated by the image generation section 44. The B-mode image data for inspection stored in the image memory 46 is read out to the display control unit 26, when necessary, so as to be displayed on the display unit 28.

The display control unit 26 displays an ultrasound image on the display unit 28 based on the B-mode image signal for inspection having been subjected to image processing by the image generation section 44.

The display unit 28 includes, for example, a display device, such as an LCD, and displays an ultrasound image under the control of the display control unit 26.

The control unit 30 controls each unit of the ultrasound diagnostic device 10 based on a command input from the operating unit 32 by the operator.

When various kinds of information, especially, information required for calculating the delay time used in the element data processing unit 22 and the phasing addition section 38 of the image generation unit 24 and information required for the element data processing in the element data processing unit 22 have been input through the operating unit 32 by the operator, the control unit 30 supplies the above-described various kinds of information input through the operating unit 32 to each unit of the transmission unit 14, the receiving unit 16, the element data storage unit 20, the element data processing unit 22, the image generation unit 24, the display control unit 26, and the like when necessary.

The operating unit 32 is used when the operator performs an input operation, and includes a keyboard, a mouse, a trackball, a touch panel, and the like.

The operating unit 32 includes an input device used when the operator inputs various kinds of information, especially, information regarding plural ultrasound elements of the probe 36 of the probe 12 used for the delay time calculation described above, sound speed in an inspection target region of the subject, a focal position of the ultrasound beam, and a transmission opening and a reception opening of the probe 36, information regarding element data processing, such as the number of sets of element data to be overlapped and an overlapping processing method, and the like when necessary.

The storage unit 34 stores various kinds of information input through the operating unit 32, especially, the above information regarding the probe 12, the sound speed, the focal position, and the transmission opening and the reception opening, information of the transmission phase for each transmission opening, the information regarding element data processing, such as the number of sets of element data to be overlapped and an overlapping processing method, and the like, or stores information required for the processing or operation of each unit controlled by the control unit 30, such as the transmission unit 14, the receiving unit 16, the element data storage unit 20, the element data processing unit 22, the image generation unit 24, and the display control unit 26, an operation program and a processing program for executing the processing or operation of each unit, and the like. It is possible to use recording media, such as a hard disk, a flexible disk, an MO, an MT, a RAM, a CD-ROM, and a DVD-ROM, as the storage unit 34.

The element data processing unit 22, the phasing addition section 38, the detection processing section 40, the DSC 42, the image generation section 44, and the display control unit 26 may be configured by a CPU and an operation program causing the CPU to perform various kinds of processing, or a hardware configuration, such as a digital circuit, may be used therefor.

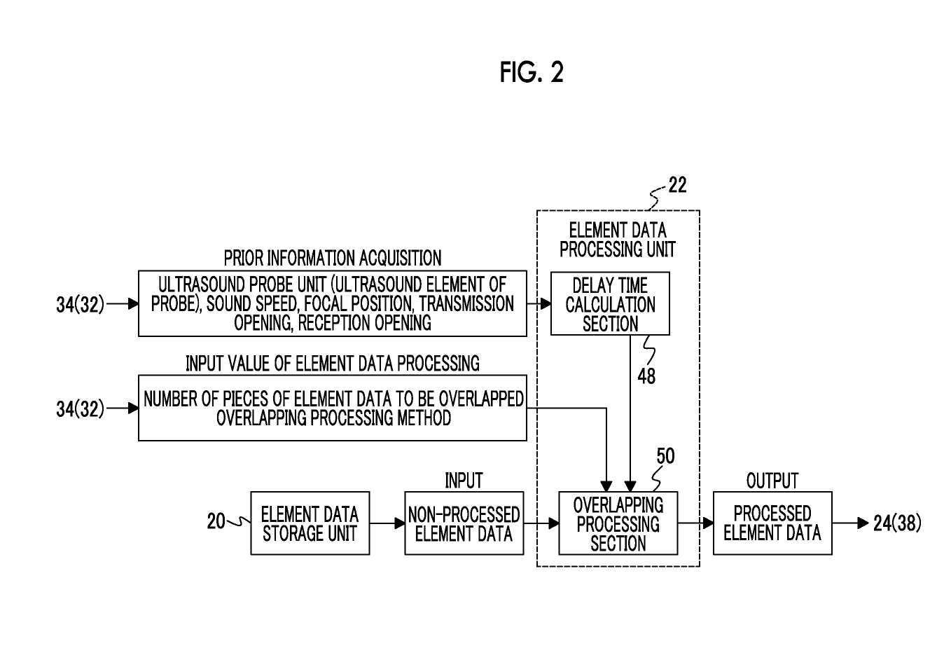

Here, the element data processing unit 22 will be described in detail. FIG. 2 is a block diagram showing the schematic configuration of an element data processing unit.

As shown in FIG. 2, the element data processing unit 22 includes a delay time calculation section 48 and an overlapping processing section 50.

The delay time calculation section 48 acquires in advance the information regarding plural ultrasound elements of the probe 36 of the probe 12, the sound speed in the inspection target region of the subject, the focal position of the ultrasound beam, the transmission opening and the reception opening of the probe 36, and the like, which has been input by operating the operating unit 32 or which is stored in the storage unit 34 after being input by operating the operating unit 32. Then, the delay time calculation section 48 calculates the delay time of element data received by each ultrasound element of the reception opening based on the geometric arrangement of the ultrasound element (transmission element) of the transmission opening that forms and transmits an ultrasound beam and the ultrasound element of the reception opening that receives an ultrasound echo from the subject by the ultrasound beam.

The overlapping processing section 50 reads two or more sets of non-processed element data generated for each of the two or more target regions, which are stored in the element data storage unit 20, based on the information regarding element data processing, such as the number of sets of element data to be overlapped and an overlapping processing method, which is input by operating the operating unit 32 or which is stored in the storage unit 34 after being input by operating the operating unit 32. Then, the overlapping processing section 50 generates processed element data by overlapping the two or more sets of non-processed element data so as to match the time on the receiving time and match the absolute position of the element of the probe that has received the ultrasound echo, based on the receiving time information of each element and the delay time calculated by the delay time calculation section 48.

Next, the element data processing performed by the element data processing unit 22 will be described in detail.

First, in a case of acquiring element data by transmitting an ultrasound beam (hereinafter, referred to as a transmission beam) from an ultrasound element for transmission (hereinafter, simply referred to as a transmission element) of the probe 36 of the ultrasound probe 12 to a subject and receiving an ultrasound echo generated by the interaction with the subject using an ultrasound element for reception (hereinafter, simply referred to as a receiving element) of the probe 36, the relationship between the transmission beam from the transmission element and the element data obtained by the receiving element will be described.

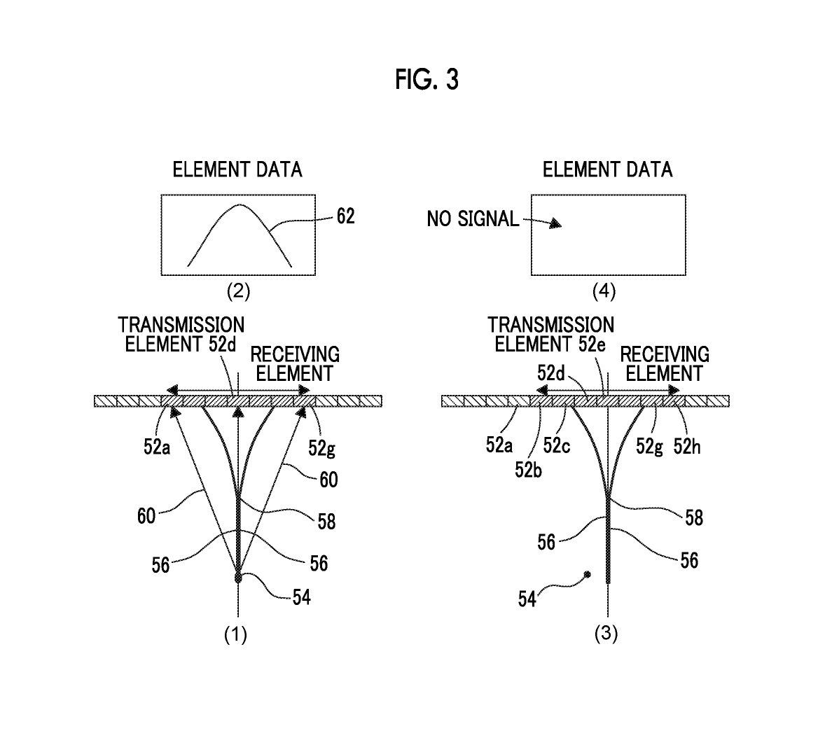

As shown in FIG. 3, element data is acquired by receiving ultrasound echoes using seven ultrasound elements (hereinafter, simply referred to as elements) 52a to 52g and seven ultrasound elements 52b to 52h as receiving elements. In the ideal case in which a transmission beam 56 to be transmitted to an inspection target region including a reflection point 54 is ideally narrowed down to an element gap or less, as shown in (1) of FIG. 3, the transmission beam 56 is transmitted by using the element 52d at the center of the elements 52a to 52g, which is located immediately above the reflection point 54 in the inspection target region, as a transmission element. When acquiring element data by receiving the ultrasound echoes using the receiving elements 52a to 52g, a focus 58 of the transmission beam 56 is located on the straight line connecting the element 52d and the reflection point 54 to each other. Accordingly, the transmission beam 56 is transmitted to the reflection point 54. As a result, an ultrasound echo reflected from the reflection point 54 is generated. The ultrasound echo from the reflection point 54 is received by the receiving elements 52a to 52g through a receiving path 60 extending at a predetermined angle, and element data 62 shown in (2) of FIG. 3 is obtained by the receiving elements 52a to 52g.

In contrast, as shown in (3) of FIG. 3, when the center of the transmission elements is shifted in the direction of the element (right direction in the diagram) by one element with respect to the reflection point 54, the transmission beam 56 is transmitted by using the element 52e adjacent to the element 52d, which is located immediately above the reflection point 54, as a transmission element, and an ultrasound echo is received by the receiving elements 52b to 52h, the reflection point 54 is not present on the straight line connecting the transmission element 52e and the focus 58 to each other in the transmission direction of the transmission beam 56. Accordingly, the transmission beam 56 is not transmitted to the reflection point 54. For this reason, since an ultrasound echo reflected from the reflection point 54 is not generated, no ultrasound echo is received by the receiving elements 52b to 52h. Therefore, as shown in (4) of FIG. 3, no element data is obtained.

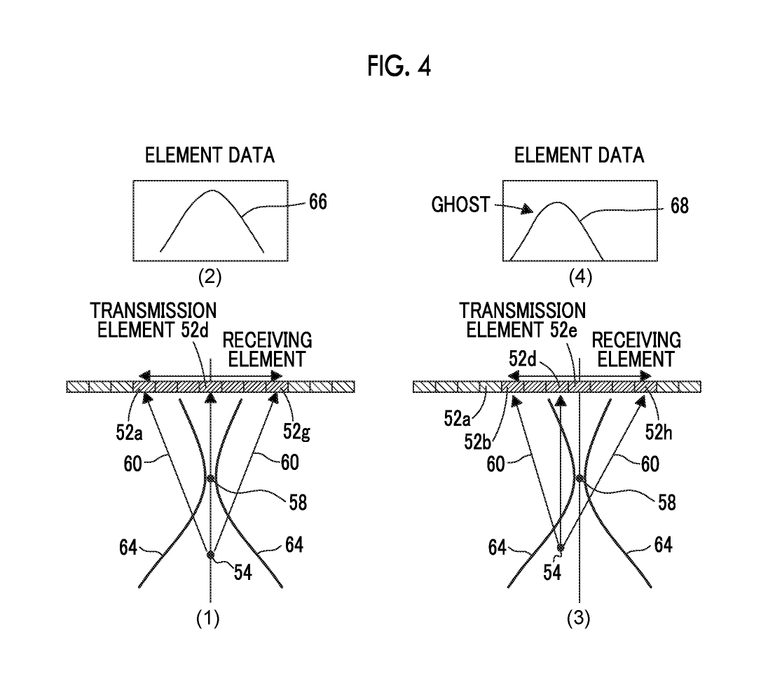

However, as shown in FIG. 4, an actual transmission beam 64 is wider than the element gap.

Here, as shown in (1) of FIG. 4, when transmitting the transmission beam 64 by using the element 52d located immediately above the reflection point 54 as a transmission element, the focus 58 is located on the straight line connecting the element 52d and the reflection point 54 to each other even if the transmission beam 64 is wide, as in the case shown in (1) of FIG. 3. Accordingly, the transmission beam 64 is reflected at the reflection point 54, and the ultrasound echo is generated. As a result, as in the case shown in (1) of FIG. 3, the ultrasound echo from the reflection point 54 is received by the receiving elements 52a to 52g through a receiving path 60 extending at a predetermined angle, and true element data 66 shown in (2) of FIG. 4 is obtained by the receiving elements 52a to 52g.

On the other hand, as shown in (3) of FIG. 4, similar to the case shown in (3) of FIG. 3, when the center of the transmission elements is shifted in the direction of the element (right direction in the diagram) by one element with respect to the reflection point 54, the transmission beam 64 is transmitted by using the element 52e adjacent to the element 52d, which is located immediately above the reflection point 54, as a transmission element, and an ultrasound echo is received by the receiving elements 52b to 52h, and the transmission beam 64 is wide. Accordingly, even if the reflection point 54 is not present on the straight line connecting the transmission element 52e and the focus 58 to each other in the transmission direction, the transmission beam 64 is transmitted to the reflection point 54. Therefore, from the reflection point 54, an ultrasound echo that is not originally present, a so-called ghost reflection signal is generated. The ghost reflection signal from the reflection point 54 is received by the receiving elements 52b to 52h through the receiving path 60 extending at a predetermined angle, and element data 68 of a ghost shown in (4) of FIG. 4 is obtained by the receiving elements 52b to 52h.

Such element data 68 of the ghost is the cause of lowering the accuracy of the ultrasound image generated from the element data.

Here, the sum (propagation distance) of a transmission path used when the transmission beam 64 reaches the reflection point 54 through the focus 58 from the transmission element 52e and a receiving path used when the ghost reflection signal reaches each of the receiving elements 52b to 52h from the reflection point 54, which is shown in (3) of FIG. 4, is longer than the sum (propagation distance) of a transmission path used when the transmission beam 64 reaches the reflection point 54 through the focus 58 from the transmission element 52d and a receiving path used when the true reflected ultrasound echo reaches each of the receiving elements 52a to 52g from the reflection point 54, which is shown in (1) of FIG. 4. Therefore, the element data 68 of the ghost shown in (4) of FIG. 4 is delayed relative to the true element data 66 shown in (2) of FIG. 4.

In the delay time calculation section 48 of the element data processing unit 22 of the present embodiment, the time difference between the true element data and the ghost element data, that is, the delay time is calculated from the geometric arrangement of the transmission element, the focus of the ultrasound beam, the reflection point of the subject, and the receiving element. Therefore, in order to calculate the delay time, information including the shape (element gap, linear, convex, and the like) of the ultrasound probe 12, the sound speed in the inspection target region of the subject, a focal position, a transmission opening, a reception opening, and the like is required. The delay time calculation section 48 acquires such information, which is input through the operating unit 32 or is stored in the storage unit 34, and calculates the delay time. For example, the delay time can be calculated from the total length (propagation distance) of the transmission path of the transmission beam reaching the reflection point through the focus from the transmission element and the receiving path of the true reflected ultrasound echo or the ghost reflection signal reaching the receiving element from the reflection point, which is calculated from the geometric arrangement of the transmission element, the focus of the ultrasound beam, the reflection point of the subject, and the receiving element, and the difference in the propagation time calculated by the sound speed.

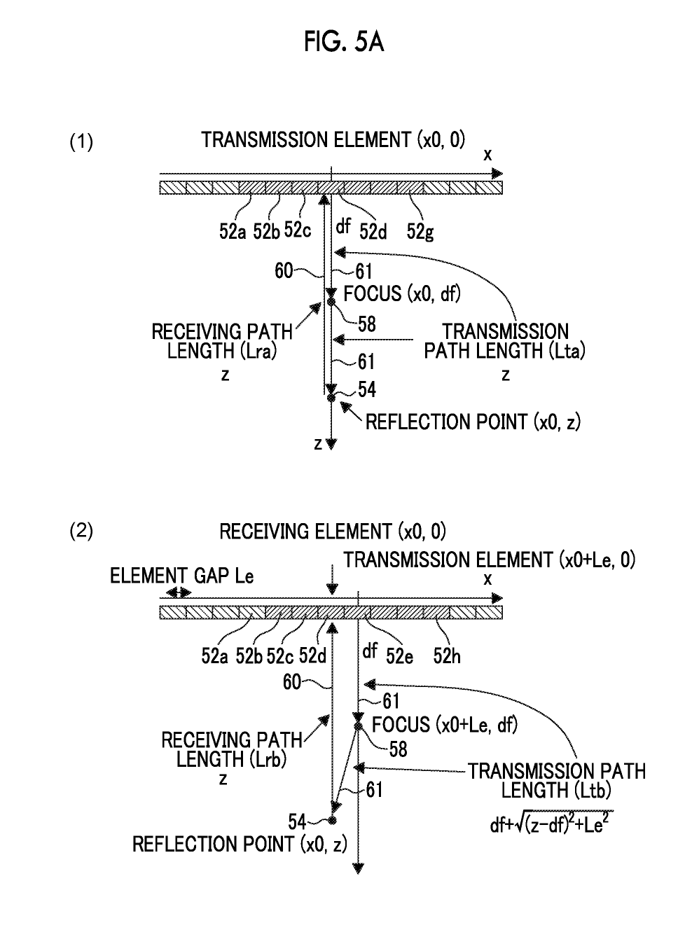

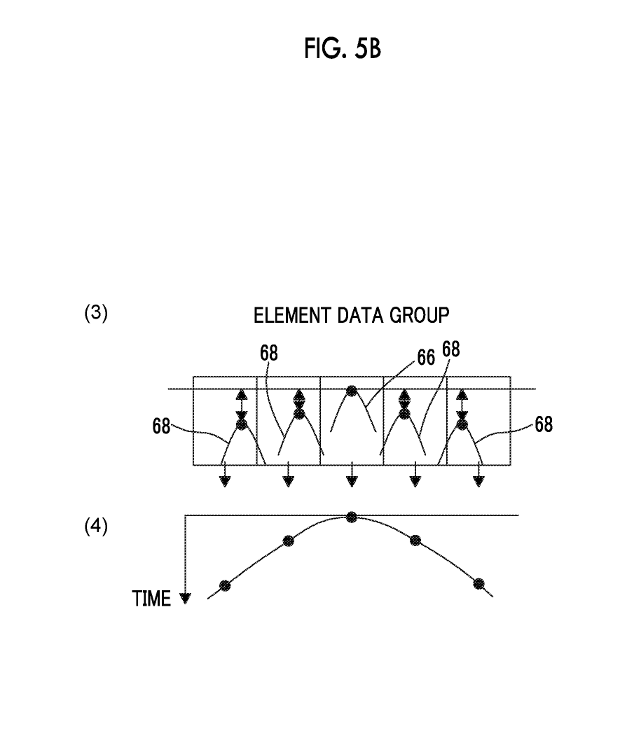

In the present embodiment, for example, as shown in FIG. 5A, it is possible to calculate the lengths of the transmission path and the receiving path of the transmission beam in the case of the true reflected ultrasound echo and the case of the ghost reflection signal.

In the case of the true reflected ultrasound echo, as shown in (1) of FIG. 5A, the transmission element 52d and the receiving element 52d (center of the receiving elements 52a to 52g) match each other, and the focus 58 and the reflection point 54 are disposed immediately below the transmission element 52d and the receiving element 52d. The position of the element 52d immediately above the reflection point 54 is set to coordinates (x0, 0) on the xy two-dimensional coordinate system, the element gap is set to Le, the position of the focus 58 is set to (x0, df), and the position of the reflection point 54 is set to coordinates (x0, z). The position of the transmission element 52d is coordinates (x0, 0), similar to the element 52d immediately above the reflection point 54. The length (transmission path length) Lta of the transmission path 61 of the transmission beam reaching the reflection point 54 through the focus 58 from the transmission element 52d and the length (receiving path length) Lra of the receiving path 60 of the true reflected ultrasound echo reaching the receiving element 52d from the reflection point 54 can be calculated from Lta=Lra=z.

Therefore, the propagation distance Lua of the ultrasound wave in the case of the true reflected ultrasound echo is Lua=Lta+Lra=2z.

In the case of the ghost reflection signal, as shown in (2) of FIG. 5A, compared with the case shown in (1) of FIG. 5A, the position of the transmission element 52e is shifted horizontally (x direction: right direction in the diagram) by one element with respect to the reflection point 54. The focus 58 is disposed immediately below the transmission element 52e, but the reflection point 54 is disposed immediately below the receiving element 52d. Assuming that the position of the receiving element 52d immediately above the reflection point 54 is the coordinates (x0, 0) on the xy two-dimensional coordinate system similar to the case shown in (1) of FIG. 5A, the element gap is Le, and the position of the reflection point 54 is the coordinates (x0, z), the position of the transmission element 52e is coordinates (x0+Le, 0), and the position of the focus 58 is coordinates (x0+Le, df). Therefore, the length (transmission path length) Ltb of the transmission path 61 of the transmission beam reaching the reflection point 54 through the focus 58 from the transmission element 52e can be calculated from Ltb=df+√{(z−df)²+Le²}. The length (receiving path length) Lrb of the receiving path 60 of the ghost reflection signal reaching the receiving element 52d from the reflection point 54 can be calculated from Lrb=z.

Therefore, the propagation distance Lub of the ultrasound wave in the case of the ghost reflection signal is Lub=Ltb+Lrb=df+√{(z−df)²+Le²}+z.

Thus, a value obtained by dividing the propagation distance Lua of the ultrasound wave, which is a sum of the distance Lta of the transmission path 61 and the distance Lra of the receiving path 60 that are determined by the geometrical arrangement shown in (1) of FIG. 5A, by the sound speed is the propagation time of the true reflected ultrasound echo. A value obtained by dividing the propagation distance Lub of the ultrasound wave, which is a sum of the distance Ltb of the transmission path 61 and the distance Lrb of the receiving path 60 that are determined by the geometrical arrangement shown in (2) of FIG. 5A, by the sound speed is the propagation time of the ghost reflection signal. The delay time is calculated from the difference between the propagation time of the true ultrasound echo when the x coordinate of the reflection point 54 and the x coordinate of the transmission element 52 (52d) are the same and the propagation time of the ghost reflection signal when the x coordinate of the reflection point 54 and the x coordinate of the transmission element 52 (52d) are shifted from each other by one element gap.

Although the geometrical model shown in FIG. 5A is a model in which the transmission path 61 extends through the focus 58, the present disclosure is not limited thereto. For example, a path that directly reaches the reflection point 54 without extending through the focus 58 may also be used.

In addition, the geometrical model shown in FIG. 5A shows the case of a linear probe. However, the present disclosure is not limited thereto, and it is possible to perform the same geometrical calculation from the shape of a probe even in other probes. For example, in the case of a convex probe, it is possible to perform the calculation in the same manner by setting the geometrical model from the radius of the probe and the angle of the element gap.

In the case of steering transmission, the delay time of the element data of the true ultrasound echo and the ghost element data therearound can be calculated from the positional relationship between the transmission element and the reflection point using the geometrical model (not shown) considering information, such as a transmission angle.

In addition, the present disclosure is not limited to the method of calculating the delay time using a geometrical model. The delay time for each set of measurement conditions may be calculated from the measurement result obtained by measuring the high-brightness reflection point in advance according to the measurement conditions of an apparatus, and the delay time may be stored in the apparatus, so that the delay time of the same measurement conditions is read.

(3) of FIG. 5B shows the element data 66 of a true signal at the center and the element data 68 of a ghost around the true signal. (4) of FIG. 5B shows an example of the delay time of the element data 68 of the ghost with respect to the element data 66 obtained from the geometrical calculation described above. (4) of FIG. 5B shows that the time of the element data 68 of the ghost signal is delayed symmetrically with respect to the element data 66 of the true signal at the center.

In addition, the delay time calculated as described above by the delay time calculation section 48 of the element data processing unit 22 can also be used for the delay correction in the phasing addition section 38.

Then, the overlapping processing section 50 of the element data processing unit 22 of the present embodiment performs overlapping processing of the element data of the true ultrasound echo and the element data of a ghost therearound using the delay time calculated by the delay time calculation section 48.

In the overlapping processing of the overlapping processing section 50, information regarding the number of sets of element data to be overlapped and an overlapping processing method when overlapping the element data is required. Such information may be input in advance through the operating unit 32, or may be stored in the storage unit 34.

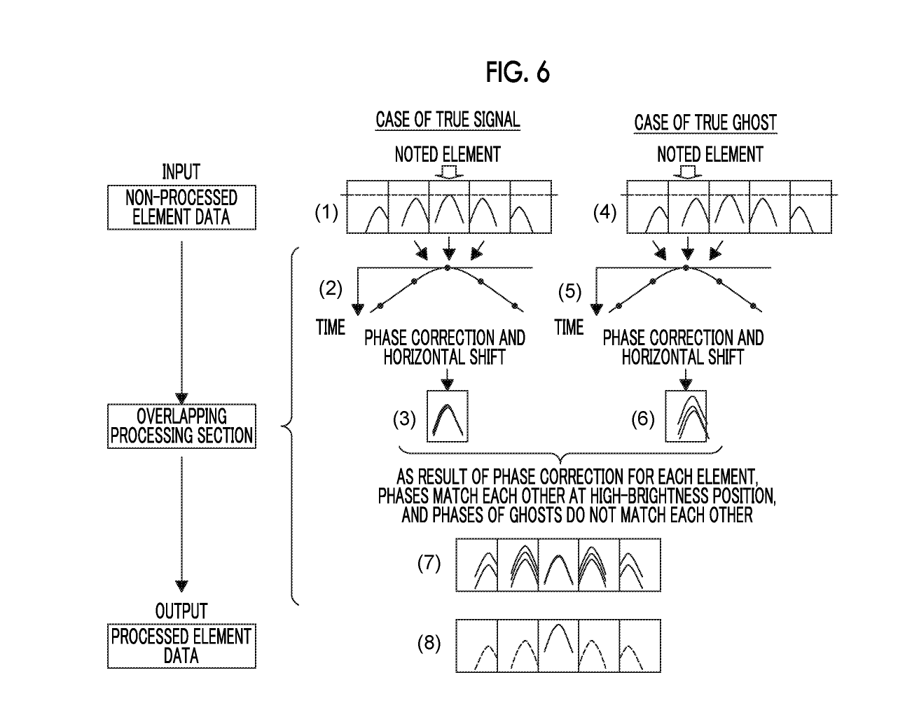

FIG. 6 shows an example of overlapping processing that is performed by the overlapping processing section 50 when the number of sets of element data is 5 and the number of sets of element data to be overlapped is 3.

(1) of FIG. 6 shows five sets of element data side by side, and shows a state in which an ultrasound beam is transmitted and the reflection signal is received for each piece of element data. The horizontal axis of the element data indicates a receiving element. The element data is displayed such that the central element at the time of transmission of the ultrasound beam is the center of the horizontal axis of the element data. The vertical axis indicates a receiving time.

In the middle element data of the five sets of element data, the reflection point is present immediately below the central element of the element data (central element of the receiving elements), that is, the central element (transmission element) at the time of transmission. Accordingly, a reflection signal from the reflection point is received. That is, this reflection signal is a true signal, and the middle element data indicates a true signal.

In the two sets of element data on each of both sides other than the middle element data, no reflection point is present immediately below the central element at the time of transmission. However, a reflection signal generated when the ultrasound beam strikes the reflection point, which is present immediately below the transmission element of the middle element data, due to the spread of the transmitted ultrasound beam, that is, a ghost is reflected. Since the time for propagation of the ultrasound wave to the reflection point increases as a distance from the true signal increases, the receiving time of a ghost is delayed from that of the true signal. In addition, the position of the receiving element where the reflection signal from the reflection point is received first is an element immediately above the reflection point, but the center of the horizontal axis of the element data is the central element at the time of transmission of the ultrasound beam. Therefore, since each piece of the element data is transmitted by shifting the central element by one element, the absolute position of the element in each piece of the element data is shifted by one element. That is, in the middle element data, the receiving element where the reflection signal from the reflection point is received first is an element in the middle. However, in the neighboring element data on both sides, the receiving element where the reflection signal from the reflection point is received first is shifted by one element from the middle element data. Therefore, the receiving element where the reflection signal from the reflection point is received first is shifted by one element to the left in the right-hand element data, and is shifted by one element to the right in the left-hand element data. Furthermore, in the element data on both ends, the receiving element where the reflection signal from the reflection point is received first is shifted by two elements from the middle element data. Therefore, the receiving element where the reflection signal from the reflection point is received first is shifted by two elements to the left in the element data on the right end, and is shifted by two elements to the right in the element data on the left end. Thus, not only is the receiving time of the ghost signal delayed from the receiving time of the true signal, but also the ghost signal differs depending on the direction of the receiving element.

(2) of FIG. 6 shows an example of the delay time of the receiving time of the five sets of element data shown in (1) of FIG. 6 with respect to the middle element data.

In the overlapping processing section 50, using the delay time shown in (2) of FIG. 6, assuming that the middle element data is a noted element, delay time correction is performed by the number of overlapping elements (in the shown example, by three elements) with the noted element at the center, and horizontal shifting is performed by the number of the noted element (in the shown example, by one element on both sides), that is, phase matching is performed, thereby overlapping non-processed element data corresponding to three elements. This data is calculated as one piece of overlapping-processed element data of the noted element.

The overlapping-processed element data of the noted element obtained as described above is shown in (3) of FIG. 6.

The element data of the noted element shown in (1) of FIG. 6 is element data of a true signal. Accordingly, if phase matching is performed by performing delay time correction and horizontal shift for non-processed element data of adjacent elements on both sides of the noted element, phase matching between the non-processed element data of the adjacent elements and the non-processed element data of the noted element is realized as shown in (3) of FIG. 6. As a result, the non-processed element data of the adjacent elements and the non-processed element data of the noted element overlap each other at a high-brightness position. Therefore, an element data value obtained, for example, by adding up these sets of element data indicates a large value (high-brightness value). For example, even if these sets of element data are averaged to calculate an average value, the element data value indicates an emphasized value (high-brightness value).

In contrast, (4) of FIG. 6 shows an example with the same element data as in (1) of FIG. 6 but with element data adjacent on the left to the middle element data, that is, a ghost being set as a noted element.

(5) of FIG. 6 is the same as (2) of FIG. 6, and shows an example of the delay time of the receiving time of the five sets of element data shown in (1) of FIG. 6 with respect to the middle element data. That is, since (1) to (4) of FIG. 6 are the same element data, the delay time of the receiving time of the five sets of element data shown in (4) of FIG. 6 with respect to the middle element data is also the same.

In the overlapping processing section 50, using the delay time shown in (5) of FIG. 6 (the same as (2) of FIG. 6), delay time correction is performed by the number of overlapping elements (in the shown example, by three elements) with the noted element at the center, and horizontal shifting is performed by the number of elements of the noted element (in the shown example, by one element on both sides), thereby overlapping non-processed element data corresponding to three elements. This data is calculated as one piece of overlapping-processed element data of the noted element.

The overlapping-processed element data of the noted element obtained as described above is shown in (6) of FIG. 6.

The element data of the noted element shown in (4) of FIG. 6 is element data of a ghost. Accordingly, even if phase matching is performed by performing delay time correction and horizontal shift for non-processed element data of adjacent elements on both sides of the noted element, phase matching between the non-processed element data of the adjacent elements and the non-processed element data of the noted element is not realized as shown in (6) of FIG. 6. As a result, the non-processed element data of the adjacent elements and the non-processed element data of the noted element do not overlap each other. For this reason, for example, even if the three sets of element data are added up, signals having inverted phases or the like are negated since the phases do not match each other. Accordingly, the sum value is not increased. In addition, for example, when the three sets of element data are averaged to calculate an average value, the average value indicates a small value.

Also for the other element data, as a noted element, the same delay time correction and horizontal shift are performed. (7) of FIG. 6 shows five overlapping states of three sets of element data adjacent to each other shown in the diagram. (8) of FIG. 6 shows a result of, for example, addition processing or averaging processing as overlapping processing that has been performed for these sets of element data.

As shown in (8) of FIG. 6, in a noted element when the coordinates of the transmission element and the reflection point shown in (1) of FIG. 6 are the same, element data of the true signal is obtained as overlapping-processed element data having a high-brightness value. On the other hand, in a total of four elements including two elements on each of both sides of the noted element, sets of element data of a ghost negate each other since element data having phases that do not match each other is added up or averaged. For this reason, the value of the overlapping-processed element data of the ghost becomes smaller than the overlapping-processed element data having a high-brightness value that is the element data of the true signal. It is possible to reduce the influence of the element data of a ghost on the element data of the true signal, or the influence of the element data of a ghost on the element data of the true signal can be made small enough to be negligible.

As the overlapping processing method in the overlapping processing section 50, not only the simple addition but also an average value or a median may be used. Alternatively, addition may be performed after the multiplication of a coefficient. Using the average value or the median can be considered to be equivalent to applying an averaging filter or a median filter in the element data level. However, instead of the averaging filter or the median filter, an inverse filter or the like used in the normal image processing may also be applied. Alternatively, element data to be overlapped may be compared, and the maximum value may be taken when the element data to be overlapped is similar, the average value may be taken when the element data to be overlapped is not similar, and the intermediate value may be taken when there is a bias in the distribution. In addition, without being limited to this, overlapping processing may be changed based on the feature amount of element data to be overlapped.

It is desirable that the number of sets of element data to be overlapped matches the spread of the beam width of the ultrasound beam. Therefore, when the beam width changes with depth, the number of sets of element data to be overlapped may also be changed according to the depth. In addition, since the beam width depends on the number of transmission openings, the number of sets of element data to be overlapped may also be changed according to the number of transmission openings. Alternatively, the number of sets of element data to be overlapped may be changed based on the feature amount, such as a brightness value of an image. In addition, the optimal number of sets of element data to be overlapped may be selected from images generated by changing the number of sets of element data to be overlapped in plural patterns.

As a result of overlapping, as described above, signals are in phase in the element data of the true signal, but signals are out of phase in the element data of a ghost. Therefore, in the element data of the ghost, as a result of overlapping processing, such as addition, signals having various phases negate each other, and this weakens the signal. Eventually, the true signal has an effective value, for example, remains as high-brightness element data, and the ghost signal has an attenuated value, for example, can be obtained as low-brightness element data.

Incidentally, in the overlapping processing described above, transmission focusing is performed by adjusting the delay time of each ultrasound element so that ultrasound beams transmitted from a set of ultrasound elements, which are a predetermined number of ultrasound elements, form one ultrasound beam. In addition, it is assumed that spherical waves converge and diverge by regarding the focus 58 formed by transmission focusing as a single sound source. However, since side lobes are secondarily formed by the transmission focusing, reflection of the side lobes is included in the reception signal. Accordingly, a ghost signal cannot be sufficiently reduced, and this becomes a cause of image quality degradation.

Therefore, in order to suppress the influence of the side lobes described above, in the present embodiment, when the overlapping processing section 50 generates processed element data by overlapping two or more sets of non-processed element data so as to match the time on the receiving time and match the absolute position of the element of the probe that has received the ultrasound echo, a phase for each transmission opening when transmitting the transmission beam is controlled so that non-processed element data having inverted phases of the respective reception signals due to a pair of openings set in advance overlap each other.

Specifically, according to the phase inversion method (KIURA Nobuyuki, “Harmonic Imaging”, Japanese Society of Radiological Technology Magazine, P 350-356), in reception waves of two transmission waves having inverted phases, fundamental wave components become waveforms having inverted phases, and secondary harmonic components become waveforms having the same phase. For this reason, by adding up the fundamental wave components and the secondary harmonic components of the two reception waves, only the secondary harmonic components are extracted. Therefore, in the present embodiment, a transmission phase for each transmission opening is determined in advance so as to extract the secondary harmonic components by adding up the reception waves of two transmission waves having inverted phases and the result is stored in the storage unit 34, and the control unit 30 controls the transmission unit 14 based on the transmission phase for each transmission opening stored in the storage unit 34.

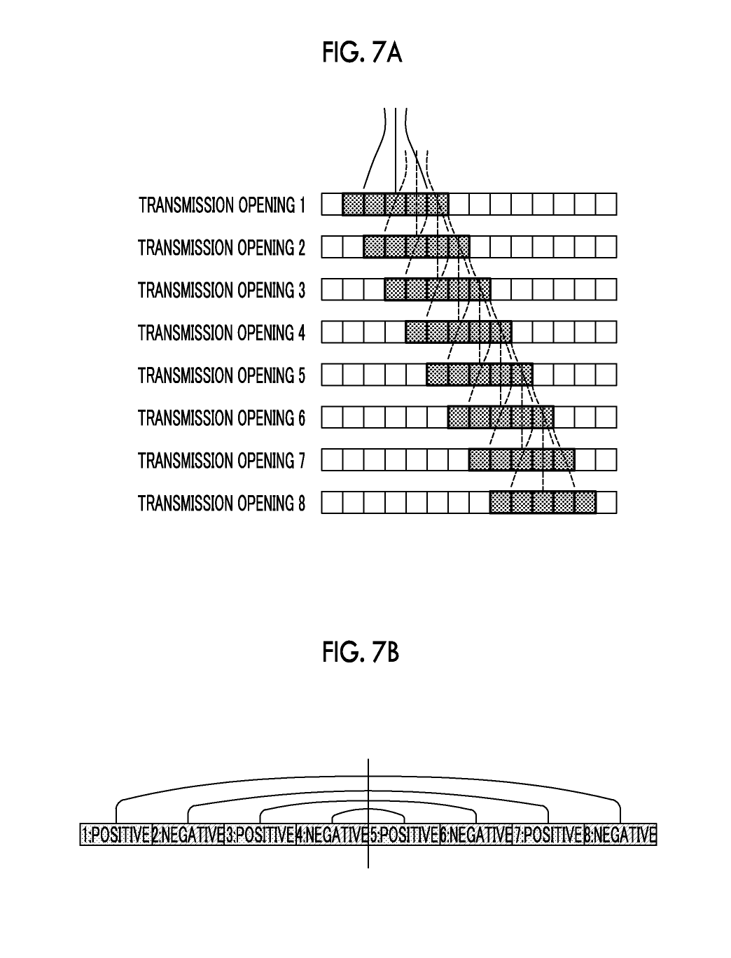

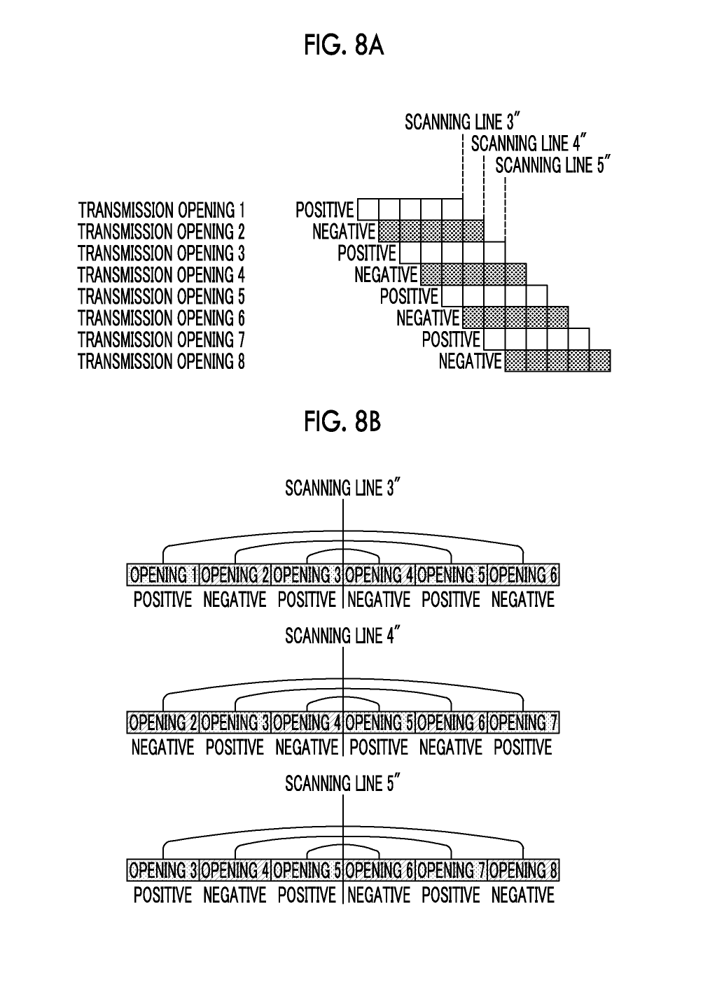

For example, as shown in FIG. 7A, when the transmission opening is formed of five ultrasound elements, an ultrasound beam is transmitted while shifting the transmission opening by one element at a time and shifting the transmission focusing line by one line at a time as indicated by transmission openings 1 to 8 in FIG. 7A. In this case, as shown in FIG. 7B, the number of transmission openings each of which is formed of five ultrasound elements is set to an even number (in FIG. 7B, eight). In addition, the transmission and reception of ultrasound beams are performed through a pair of positive and negative transmission openings having inverted phases, and a pair of transmission openings are determined in advance so as to form a pair of positive and negative transmission openings having phases that are inverted at a transmission opening where the sound pressure from the noted point is the same, thereby transmitting an ultrasound beam. More specifically, as shown in FIG. 8A, transmission and reception of ultrasound beams are performed corresponding to scanning lines 1 to 8 while shifting the transmission opening by one element. In this case, as shown in FIG. 8A, openings located symmetrically with respect to the scanning line are assumed to be an opening of the positive phase and an opening of the negative phase, and the phases of the adjacent transmission openings are assumed to be positive and negative alternately. Then, by performing overlapping processing using six sets of element data corresponding to the scanning lines 1 to 8 that have been acquired as described above, element data corresponding to each scanning line after the overlapping processing is generated. For example, element data corresponding to a scanning line 3″, a scanning line 4″, and a scanning line 5″ is generated by the overlapping processing as shown in FIG. 8B. Therefore, since the phases of the openings located symmetrically with respect to the scanning line are positive and negative, harmonic components are added up after being extracted by the overlapping processing.

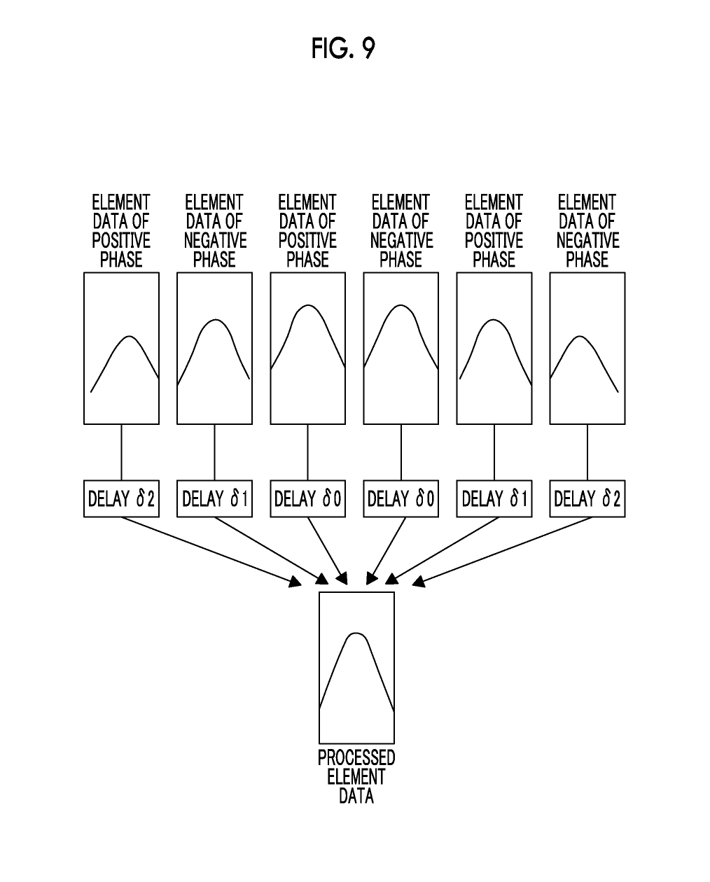

That is, as shown in FIG. 9, in a pair of positive and negative openings having inverted phases where the sound pressure from the noted point is the same, element data obtained by reception at the openings is positively and negatively phase-inverted element data. Accordingly, by adding up the element data for each delay time (δ0 to δ2), only the secondary harmonic components are extracted. Therefore, the influence of side lobes is suppressed by performing the overlapping processing using only the secondary harmonic components.

Subsequently, the operation and effect of the ultrasound diagnostic device of the present disclosure and a method of generating an ultrasound image will be described.

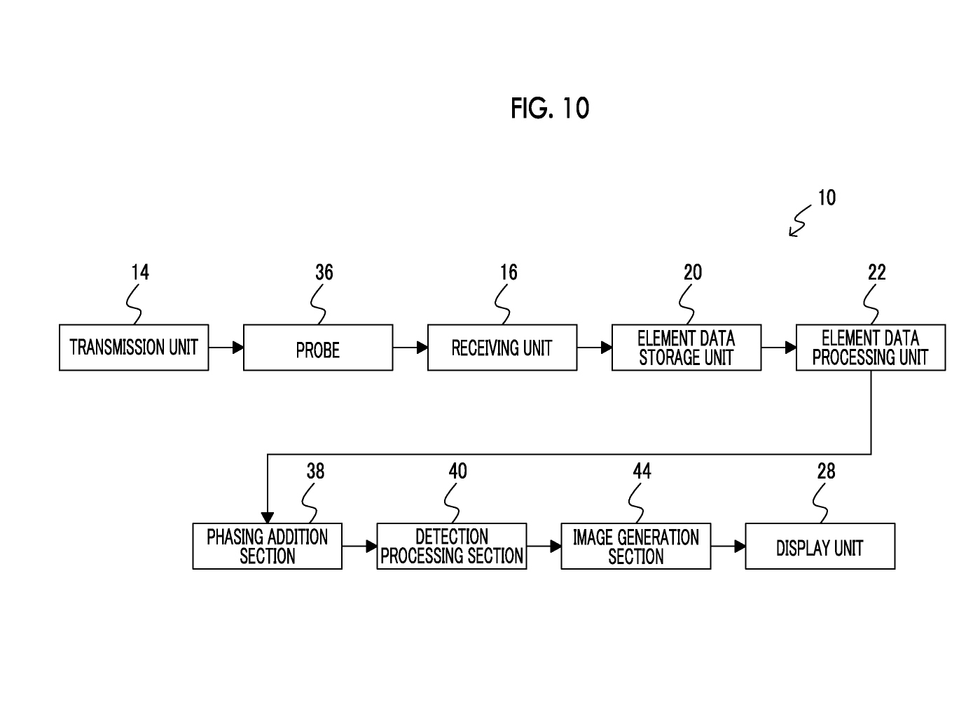

FIG. 10 is a block diagram showing the main part of the ultrasound diagnostic device according to the present embodiment shown in FIG. 1 along the process flow.

As shown in FIG. 1, when the operator brings the ultrasound probe 12 into contact with the surface of the subject to start measurement, an ultrasound beam is transmitted from the probe 36 according to the driving signal supplied from the transmission unit 14 as shown in FIG. 10. In this case, the control unit 30 controls the transmission unit 14 based on the transmission phase for each transmission opening stored in advance in the storage unit 34, so that positive and negative ultrasound beams are transmitted by inverting the phases in a pair of transmission openings where the sound pressure from the noted point is the same.

The ultrasound echo generated by the interaction between the transmitted ultrasound beam and the subject is received by the probe 36, and an analog element signal is output as a reception signal.

The receiving unit 16 amplifies the analog element signal and supplies the amplified analog element signal to the A/D conversion unit 18, and the A/D conversion unit 18 converts the analog element signal into digital element data and supplies the digital element data to the element data storage unit 20 in order to store the digital element data.

In the element data processing unit 22, the delay time calculation section 48 (FIG. 2) calculates the delay time (for example, the same in both (2) and (5) of FIG. 6) of the non-processed element data of the surrounding ghost signal with respect to the non-processed element data of the true signal from the geometric arrangement of the transmission element, the focus, the reflection point, and the receiving element, the sound speed in the inspection target region of the subject that is input and set in advance, and the like (for example, calculated using the geometrical model in FIGS. 5A and 5B).

Then, the element data processing unit 22 reads non-processed element data from the element data storage unit 20, and sets element data to be processed for a noted element, and the overlapping processing section 50 (FIG. 2) calculates processed element data by overlapping the noted element and non-processed element data around the noted element with each other by phase matching using the delay time calculated by the delay time calculation section 48. Accordingly, emphasized processed element data is obtained in the case of non-processed element data including a true signal, and attenuated processed element data is obtained in the case of non-processed element data of a ghost. In addition, an ultrasound beam is transmitted by inverting the phases of a pair of transmission openings where the sound pressure from the noted point is the same, and the overlapping processing section 50 performs overlapping processing. Then, processed element data is obtained in which only the secondary harmonic components have been extracted by adding up the element data received in a pair of openings having inverted phases.

The element data processing unit 22 supplies the processed element data obtained as described above to the phasing addition section 38 of the image generation unit 24.

The phasing addition section 38 of the image generation unit 24 generates reception data (acoustic ray signal) by performing reception focusing processing on the element data, and supplies the generated reception data to the detection processing section 40. The detection processing section 40 generates a B-mode image signal by processing the acoustic ray signal. The B-mode image signal is subjected to raster conversion by the DSC 42 and to image processing by the image generation section 44, thereby generating an ultrasound image. The generated ultrasound image is stored in the image memory 46, and the ultrasound image is displayed on the display unit 28 by the display control unit 26.

Thus, in the ultrasound diagnostic device 10 according to the embodiment of the present disclosure, the element data processing unit 22 generates an ultrasound image using the processed element data in which a ghost signal is weakened and the true signal is emphasized. Accordingly, it is possible to obtain an ultrasound image having improved azimuth resolution and contrast resolution.

In addition, since only the secondary harmonic components are extracted and processed by performing the overlapping processing by controlling the transmission unit 14 to transmit ultrasound beams having inverted phases in a pair of transmission openings where the sound pressure from the noted point is the same, it is possible to suppress the influence of side lobes.

In a conventional phase inversion method of extracting and imaging secondary harmonic components, the time resolution is reduced since the number of frames for imaging is halved by two transmissions. In the present embodiment, however, since the element data based on a transmission wave having a positive phase and the element data based on a transmission wave having a negative phase are made to overlap each other in the overlapping processing, the frame rate is not reduced. In addition, since completely the same processing as normal overlapping processing is performed, it is possible to realize overlapping processing using secondary harmonic components.

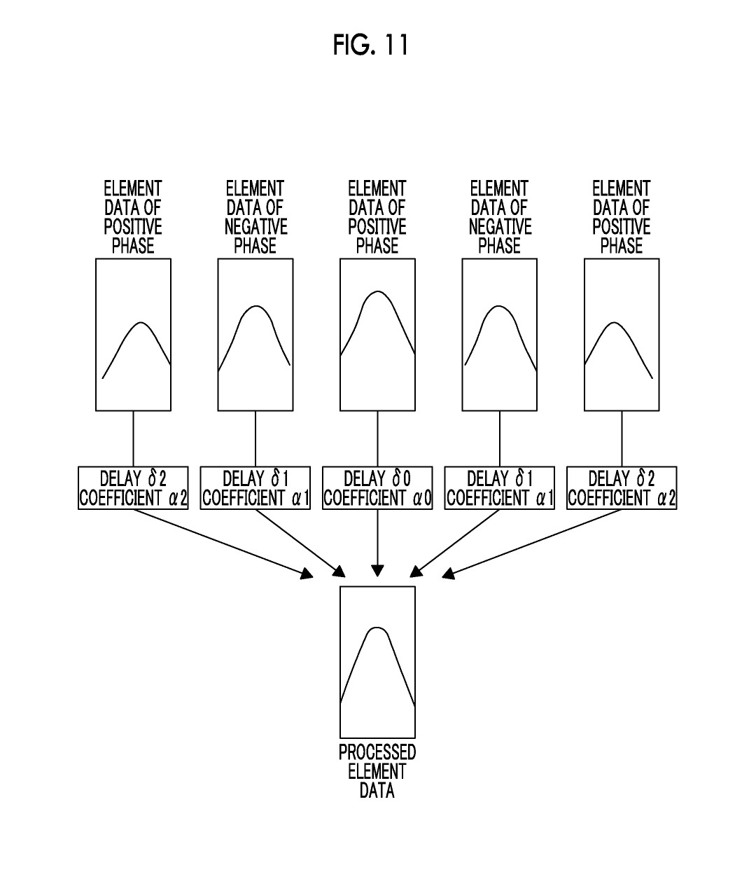

Incidentally, in the above embodiment, a case in which the number of transmission openings is even has been described as an example. However, the number of transmission openings may be odd. When the number of transmission openings is even, it is possible to adjust the sound pressure from the noted point by setting the transmission openings, which are located symmetrically with respect to the scanning line, as an opening of a positive phase and an opening of a negative phase. However, when the number of transmission openings is an odd number, the sound pressure from the noted point varies. In this case, therefore, as shown in FIG. 11, coefficients (α0 to α2) are determined in advance so as to make the sound pressure from the noted point uniform based on the profile of the ultrasound beam, and overlapping processing is performed using the determined coefficients. As a result, as in the embodiment described above, it is possible to suppress the influence of side lobes by extracting only the secondary harmonic components.

In the embodiment described above, reception data (acoustic ray signal) is generated by generating processed element data by performing overlapping processing on non-processed element data and then performing phasing addition (receiving focus) on the processed element data. However, processed reception data (acoustic ray signal) may be generated by generating non-processed reception data (acoustic ray signal) by performing phasing addition (receiving focus) on non-processed element data and then performing overlapping processing on the non-processed element data. Even if the order of processing is changed in this manner, it is possible to obtain completely the same reception data (acoustic ray signal) as in the embodiment described above.

In the ultrasound diagnostic device 10 according to the embodiment of the present disclosure, the sound speed in the inspection target region of the subject, which is required when the delay time calculation section 48 of the element data processing unit 22 calculates a delay time and when calculating the delay time used in the phasing addition section 38, is given. However, the present disclosure is not limited to this. When the sound speed in the inspection target is not known, an optimal sound speed may be calculated and set using various kinds of known method.

In addition, the processes performed by the respective units in the embodiment described above may be distributed as a program by being stored in various storage media.

In addition, the configuration, operation, and the like of the ultrasound diagnostic device 10 described in the present embodiment are examples, and it is needless to say that these can be changed according to the circumstances within the scope not deviating from the spirit of the present disclosure.

All documents, patent applications, and technical standards described in this specification are incorporated in this specification by reference to the same extent as when the incorporation of individual documents, patent applications, and technical standards by reference is described specifically and individually.

What is claimed is:

1. An ultrasound diagnostic device, comprising:
a probe including a plurality of elements for generating ultrasound waves and receiving ultrasound waves reflected from an inspection target;
a transmission unit that transmits ultrasound waves from the plurality of elements such that an ultrasound beam is transmitted to the inspection target by determining a group of a predetermined number of elements of the probe as openings, determining in advance an opening of a positive phase and an opening of a negative phase corresponding to the opening of the positive phase and inverting phases thereof;
a receiving unit that receives an ultrasound echo signal generated by interaction between the ultrasound beam and the inspection target, through the plurality of elements of the probe;
an element data storage unit that stores two or more sets of element data including receiving time information in each element, the two or more sets of element data being generated by transmitting the ultrasound beam from openings having different phases and having different elements at centers, in each of at least two or more overlapping target regions in the inspection target using the transmission unit, and receiving the ultrasound echo signal generated for each of the at least two or more overlapping target regions in the inspection target by the ultrasound beam, using the receiving unit; and
a generation unit that generates an acoustic ray signal in one scanning line by overlapping the element data stored in the element data storage unit.

2. The ultrasound diagnostic device according to claim 1,
wherein the transmission unit transmits ultrasound waves from the plurality of elements by determining the number of the openings as an even number, determining in advance openings that are located symmetrically with respect to the scanning line as the opening of the positive phase and the opening of the negative phase, inverting the phases thereof and transmitting the ultrasound beam from each of the openings.

3. The ultrasound diagnostic device according to claim 1,
wherein the transmission unit transmits ultrasound waves from the plurality of elements by determining the number of the openings as an odd number, inverting the phases thereof and transmitting the ultrasound beam from each of the opening of the positive phase and the opening of the negative phase, and
the generation unit generates the acoustic ray signal by overlapping the element data stored in the element data storage unit based on the receiving time information and a coefficient that is set in advance for each opening such that sound pressure from a noted point is the same.

4. The ultrasound diagnostic device according to claim 1,
wherein the generation unit includes:
a delay time calculation section that calculates a delay time of the two or more sets of the element data; and
an overlapping processing section that overlaps two or more sets of non-processed element data so that times thereof match on a receiving time and absolute positions of the elements of the probe that have received the ultrasound echo signal match, based on the delay time calculated by the delay time calculation section, the receiving time information, and positions of the elements of the probe that has received the ultrasound echo signal.

5. The ultrasound diagnostic device according to claim 1,
wherein the transmission unit transmits ultrasound waves from the plurality of elements such that the opening of the positive phase and the opening of the negative phase adjacent to each other alternately transmit ultrasound beams.

6. An acoustic ray signal generation method of an ultrasound diagnostic device including a transmission unit that transmits ultrasound waves from a plurality of elements of a probe for generating ultrasound waves and receiving ultrasound waves reflected from the inspection target, such that the ultrasound beam is transmitted to an inspection target by determining a group of a predetermined number of the elements of the probe as openings, determining in advance an opening of a positive phase and an opening of a negative phase corresponding to the opening of the positive phase and inverting phases thereof, a receiving unit that receives an ultrasound echo signal generated by interaction between the ultrasound beam and the inspection target, through the plurality of elements of the probe, and in an element data storage unit, the method comprising:
transmitting, by the transmission unit, an ultrasound beam from openings having different phases and having different elements at centers, in each of at least two or more overlapping target regions in the inspection target;
receiving, by the receiving unit, an ultrasound echo signal generated for each of the at least two or more overlapping target regions in the inspection target;
storing two or more sets of element data generated by receiving the ultrasound echo signal in the element data storage unit, the two or more set of element data including receiving time information in each element; and
generating an acoustic ray signal in one scanning line by overlapping the element data stored in the element data storage unit.

7. The acoustic ray signal generation method according to claim 6,
wherein the transmission of the ultrasound beam by the transmission unit further includes transmitting ultrasound waves from the plurality of elements by determining the number of the openings as an even number, determining in advance openings that are located symmetrically with respect to the scanning line as the opening of the positive phase and the opening of the negative phase, inverting the phases thereof and transmitting the ultrasound beam from each of the openings.

8. The acoustic ray signal generation method according to claim 6,
wherein the transmission of the ultrasound beam by the transmission unit further includes transmitting ultrasound waves from the plurality of elements by inverting phases of the opening of the positive phase and the opening of the negative phase in the transmission unit having an odd number of the openings, and
the generation of the acoustic ray signal further includes generating the acoustic ray signal by overlapping the element data stored in the element data storage unit based on the receiving time information and a coefficient that is set in advance for each opening such that sound pressure from a noted point is the same.

9. The acoustic ray signal generation method according to claim 6,
wherein the generation of the acoustic ray signal further includes:
calculating a delay time of the two or more sets of element data; and
overlapping two or more sets of non-processed element data so that times thereof match on a receiving time and absolute positions of the elements of the probe that have received the ultrasound echo signal match, based on the calculated delay time, the receiving time information, and positions of the elements of the probe that have received the ultrasound echo signal.

10. The acoustic ray signal generation method according to claim 6,
wherein the transmission of the ultrasound beam further includes transmitting ultrasound waves from the plurality of elements such that the opening of the positive phase and the opening of the negative phase adjacent to each other alternately transmit ultrasound beams.

11. A non-transitory storage medium that stores a program causing a computer to execute an acoustic ray signal generation processing of an ultrasound diagnostic device including a transmission unit that transmits ultrasound waves from a plurality of elements of a probe for generating ultrasound waves and receiving ultrasound waves reflected from the inspection target, such that the ultrasound beam is transmitted to an inspection target by determining a group of a predetermined number of the elements of the probe as openings, determining in advance an opening of a positive phase and an opening of a negative phase corresponding to the opening of the positive phase and inverting phases thereof, a receiving unit that receives an ultrasound echo signal generated by interaction between the ultrasound beam and the inspection target, through the plurality of elements of the probe, and in an element data storage unit,
the processing comprising:
transmitting, by the transmission unit, an ultrasound beam from openings having different phases and having different elements at centers, in each of at least two or more overlapping target regions in the inspection target;
receiving, by the receiving unit, an ultrasound echo signal is generated for each of the at least two or more overlapping target regions in the inspection target;
storing two or more sets of element data generated by receiving the ultrasound echo signal in the element data storage unit, the two or more sets of element data including receiving time information in each element; and
generating an acoustic ray signal in one scanning line by overlapping the element data stored in the element data storage unit.

12. The non-transitory storage medium according to claim 11,
wherein the transmission of the ultrasound beam by the transmission unit further includes transmitting ultrasound waves from the plurality of elements by determining the number of the openings as an even number, determining in advance openings that are located symmetrically with respect to the scanning line as the opening of the positive phase and the opening of the negative phase, inverting phases thereof and transmitting the ultrasound beam from each of the openings.

13. The non-transitory storage medium according to claim 11,
wherein the transmission of the ultrasound beam by the transmission unit further includes transmitting ultrasound waves from the plurality of elements by inverting phases of the opening of the positive phase and the opening of the negative phase of the transmission unit having an odd number of the openings, and
the generation of the acoustic ray signal further includes generating the acoustic ray signal by overlapping the element data stored in the element data storage unit based on the receiving time information and a coefficient that is set in advance for each opening such that sound pressure from a noted point is the same.

14. The non-transitory storage medium according to claim 11,
wherein the generation of the acoustic ray signal further includes:
calculating a delay time of the two or more sets of element data; and
overlapping two or more sets of non-processed element data so that times thereof match on a receiving time and absolute positions of the elements of the probe that have received the ultrasound echo signal match, based on the calculated delay time, the receiving time information, and positions of the elements of the probe that have received the ultrasound echo signal.

15. The non-transitory storage medium according to claim 11,
wherein the transmission of the ultrasound beam further includes transmitting ultrasound waves from the plurality of elements such that the opening of the positive phase and the opening of the negative phase adjacent to each other alternately transmit ultrasound beams.
